(19) United States
(12) Patent Application Publication
(10) Pub. No.: US 2023/0410587 A1
(43) Pub. Date: Dec. 21, 2023

(54) MONITORING HEALTH POLICY COMPLIANCE FOR PROTECTING CONSUMERS HEALTH AGAINST CONTAGIOUS INFECTIOUS DISEASES WHEN USING PRODUCT VENDING MACHINES

(71) Applicant: Evoca S.P.A, Valbrembo (BG) (IT)

(72) Inventor: Alberto Capobianco, Valbrembo (IT)

(21) Appl. No.: 18/251,612

(22) PCT Filed: Nov. 5, 2021
(86) PCT No.: PCT/IB2021/060274

§ 371 Date: May 3, 2023

(30) Foreign Application Priority Data
Nov. 5, 2020 (EP) 20206010.9
Oct. 25, 2021 (IT) 102021000027371

(51) Int. Cl. G07F 9/00; G07F 9/02; G06V 40/10; A61B 5/01

(52) U.S. Cl. CPC G07F 9/009; G07F 9/023; A61B 2503/12; A61B 5/01; G06V 40/10

(57) ABSTRACT

A product vending machine comprising a monitoring system designed to monitor whether consumers in a consumer area of the product vending machine comply with one or more stored health policies for protecting consumers' health against contagious infectious diseases and containing recommendations on one or more of interpersonal distance, body temperature and use of respiratory personal protective equipment. If one or more consumers in the consumer area of the product vending machine are determined to fail to comply with the stored health policies, the monitoring system is further designed to implement one or both of informing the consumers in the consumer area of the product vending machine that one or more of the stored health policies fail to be complied with by one or more of the consumers in the consumer area of the product vending machine, and modify operation of the product vending machine so as to cause the consumers in the consumer area of the product vending machine to become aware that one or more of the stored health policies fail to be complied with by one or more of the consumers in the consumer area of the product vending machine.

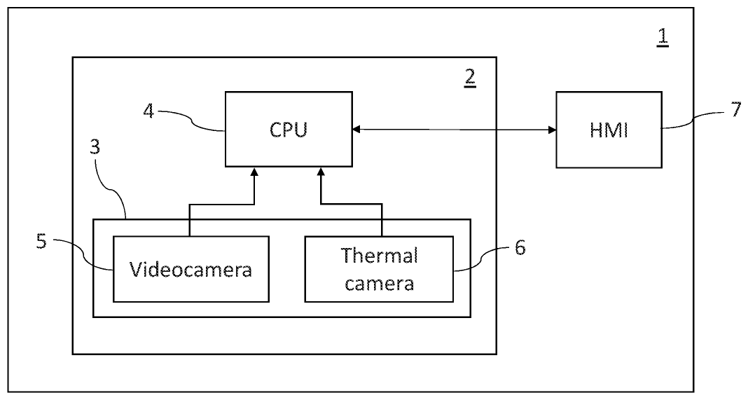

### CROSS-REFERENCE TO RELATED APPLICATIONS

This patent application claims priority to European patent application no. 20206010.9 filed on 5 Nov. 2020 and Italian patent application no. 102021000027371 filed on 25.10.2021.

### TECHNICAL FIELD OF THE INVENTION

The present invention relates in general to product vending machines, and in particular to monitoring health policy compliance for protecting consumers' health against contagious infectious diseases when using product vending machines.

The present invention finds application in product vending machines dispensing products of any kind and in any state of matter, in particular solid or liquid food products, packaged or prepared at the moment on demand.

The present invention finds advantageous application in beverage vending machines dispensing hot or cold, packaged or so-called instant beverages, i.e., prepared on demand from liquid and/or powdered ingredients, to which the following disclosure will explicitly refer without losing generality.

The present invention may, however, find advantageous application in product vending machines dispensing products of a different kind, such as electronic products, gadgets, personal protective equipment, etc.

### STATE OF THE ART

As known, in the field of vending machines, design is increasingly focusing on the development and adoption of technologies that increase interactivity between consumers and vending machines. For this reason, the Human-Machine Interfaces (HMI) of vending machines, e.g. dispensing instant beverages, are continuously evolving to allow consumers to interact with vending machines in an increasingly sophisticated way from different perspectives.

KR 102 134 903 B1 discloses an infection prevention certification kiosk comprising a housing with a hand inlet; a hand sanitizer dispenser installed in the hand inlet to automatically discharge a disinfectant solution when a user's hand is inserted in the hand inlet; a first body temperature measuring unit installed in the housing to measure body temperature through a thermal image of the user; a second body temperature measuring unit installed in the housing to measure the user body temperature in a non-contact manner; a certification issuance unit issuing infection prevention certification stickers for access and passage of multi-use facilities; and a control unit executing an output unit that outputs a warning message and an infection prevention authentication control program to control each unit. Therefore, it is possible to issue the infection prevention certification sticker in an unmanned manner, accurately measure body temperature with a double body temperature check, and share the authentication sticker, thereby smoothly controlling access of multi-use facilities.

CN 111 429 652 A discloses an intelligent vending machine with a respiratory disease screening function. The intelligent vending machine comprises a three-mode camera connected with an image/information processing module, and thermal imaging and depth picture acquisition are completed when a user uses automatic vending. The image/information processing module extracts the body temperature and the respiratory waveform of the user for respiratory disease screening, and transmits a screening result to a server and the mobile phone of the user. The body temperature and breath are detected and screened during use of the vending machine and abnormal information is sent to the user and related management departments as soon as possible. Infectious respiratory diseases can be prevented from spreading in public places to a certain extent and respiratory infection diseases of people in the public places can be screened while commodity purchasing of the user is not affected.

Marco Cristani et al., “The Visual Social Distancing Problem”, Arxiv.org, Cornell University Library, 201 Olin Library Cornell University Ithaca, NY 14853, (20200511) discloses a Visual Social Distancing (VSD) problem, defined as the automatic estimation of the inter-personal distance from an image, and the characterization of the related people aggregations. It is discussed how VSD relates with previous literature in Social Signal Processing and indicate which existing Computer Vision methods can be used to manage such problem.

US 2007/013493 A1 discloses a multi-functional monitor that utilizes a CCD camera to record an image of a head of a person. The image is then transmitted to an image processor (or a human-image recognition processor) for producing pixels of luminosity data of the image. Eventually, the pixel data are put into a central processing unit (CPU) wherein they are checked against a normal data for consistency. If they are not consistent or the person is determined to have an exceedingly high body temperature, the central processing unit (CPU) will control a driver to start a video recorder. Meanwhile, a relay will be activated to turn on a speaker and an LCD for warning the person and showing the body-temperature reading of the person. If the person continues to force in, the central processing unit (CPU) will notify a police authority through an automatic alarm system. It also warns the person with a flash light.

CN 111 311 799 A discloses a gate control system based on intelligent identification. The gate control system comprises a photoelectric detection switch, a video acquisition module, an image recognition module, an infrared body temperature detection module, a counting module, an information processor, a voice module and a gate device. When epidemic situations need to be prevented and controlled, the control system can intelligently carry out recognition and only allow people who correctly wear masks and have normal body temperatures to enter public places such as shopping malls, supermarkets, exhibition halls, scenic spots and entertainment venues; the number of people entering the public places is limited, so that the situation that people are too dense is avoided. Therefore, potential safety hazards of mutual infection between people are eliminated.

### OBJECT AND SUMMARY OF THE INVENTION

The Applicant has observed that, in the current historical period, in which it is of vital importance to counter the spread of viruses such as Sars-CoV-2, also commonly

1-10. (canceled)

11. A product vending machine, comprising:
a monitoring system designed to:
monitor whether consumers in a consumer area of the product vending machine comply with one or more stored health policies for protecting consumers' health against contagious infectious diseases and containing recommendations on one or more of interpersonal distance, body temperature and use of respiratory personal protective equipment that consumers have to comply with in the consumer area of the product vending machine; and
if one or more consumers in the consumer area of the product vending machine are determined to fail to comply with one or more of the stored health policies, implement one or both of:
informing the consumers in the consumer area of the product vending machine that one or more of the stored health policies fail to be complied with by one or more of the consumers in the consumer area of the product vending machine; and
modifying operation of the product vending machine to cause the consumers in the consumer area of the product vending machine to become aware that one or more of the health policies fail to be complied with by one or more of the consumers in the consumer area of the product vending machine.

12. The product vending machine of claim 11, wherein the monitoring system comprises:
a sensory system designed to output an output such as to allow to determine whether the consumers in the consumer area of the product vending machine comply with one or more of the stored health policies; and
electronic storage and processing resources designed to store data representative of one or more health policies that consumers in the consumer area of the product vending machine have to comply with, and to communicate with the sensory system to receive and process the output of the sensory system based on the stored health policies in order to determine whether the consumers in the consumer area of the product vending machine comply with one or more of the stored health policies.

13. The product vending machine of claim 12, wherein the sensory system comprises an electronic digital image/video capture device arranged to capture digital images/videos of the consumer area of the product vending machine;
the electronic storage and processing resources are further designed to receive digital images/videos from the electronic digital image/video capture device and to process the received digital images/videos based on the stored health policies in order to determine whether the consumers in the consumer area of the vending machine comply with one or more of the stored health policies.

14. The product vending machine of claim 13, wherein the electronic storage and processing resources are further designed to process the received digital images/videos to:
identify the consumers in the consumer area of the product vending machine;
determine one or both of the interpersonal distance of the consumers in the consumer area of the product vending machine and whether the consumers in the consumer area of the product vending machine are wearing respiratory personal protective equipment; and
determine whether the consumers in the consumer area of the product vending machine comply with the stored health policies relating to one or both of the interpersonal distance and the wearing of respiratory personal protective equipment.

15. The product vending machine of claim 12, wherein the sensory system comprises a contactless body temperature measuring device operable to measure, and output an output indicative of, the body temperatures of the consumers in the consumer area of the product vending machine;
the electronic storage and processing resources are configured to:
receive and process the output of the contactless body temperature measuring device to determine the body temperatures of the consumers in the consumer area of the product vending machine; and
determine whether the body temperatures of the consumers in the consumer area of the product vending machine comply with the stored health policy relating to the body temperature.

16. The product vending machine of claim 11, wherein the electronic storage and processing resources are further designed to cause the consumers in the consumer area of the product vending machine to be informed that one or more of the health policies fail to be complied with by one or more of the consumers in the consumer area of the product vending machine by displaying, on a display of the product vending machine, either a video feed from the electronic digital image/video capture device or a graphical representation of the consumers in the consumer area of the product vending machine constructed based on the captured digital images/videos, both with highlighting or graphical indications of the consumers who fail comply with the stored health policies, or even a simplified graphical representation containing one or more of the following information:
number of consumers in the consumer area of the product vending machine,
presence of consumers in the consumer area of the product vending machine who fail to comply with the stored health policies,
number of consumers in the consumer area of the product vending machine who fail to comply with the stored health policies,
number of consumers in the consumer area of the product vending machine who fail to maintain the recommended interpersonal distance,
number of consumers in the consumer area of the product vending machine who fail to wear a respiratory personal protective equipment, and
number of consumers in the consumer area of the product vending machine the body temperature of which is not in compliance with the stored health policies.

17. The product vending machine of claim 11, wherein the electronic storage and processing resources are further designed to modify operation of the product vending machine by either limiting or inhibiting operation of the product vending machine as long as one or more of the stored health policies fail to be complied with by one or more of the consumers in the consumer area of the product vending machine.

18. The product vending machine of claim 11, wherein the electronic storage and processing resources are further designed to:
determine a degree of severity of failure of the consumers in the consumer area of the product vending machine to comply with the stored health policies based on a stored assessment criterion; and
determine whether to inform the consumers in the consumer area of the product vending machine that one or more of the health policies fail to be complied with by one or more of the consumers in the consumer area of the product vending machine either through a display of the product vending machine or by modifying operation of the product vending machine based on the degree of severity of failure of the consumers in the consumer area of the product vending machine to comply with the stored health policies.

19. The product vending machine of claim 18, wherein the electronic storage and processing resources are further designed to display on a display of the product vending machine different warnings to consumers in the consumer area of the product vending machine depending on the degree of severity of failure of the consumers in the consumer area of the product vending machine to comply with the stored health policies and to modify operation of the product vending machine if the degree of severity of failure of the consumers in the consumer area of the product vending machine to comply with the stored health policies is even higher.

20. Software loadable in the electronic storage and processing resources of the product vending machine of claim 11 and designed to cause, when run, the electronic storage and processing resources to become configured as claimed in claim 11.
